(19) United States
(12) Patent Application Publication
(10) Pub. No.: US 2024/0180921 A1
(43) Pub. Date: Jun. 6, 2024

(54) CDK8/19 INHIBITORS FOR THE TREATMENT OF CYTOKINE STORM

(71) Applicants: University of South Carolina, Columbia, SC (US); Senex Biotechnology, Inc., Columbia, SC (US)

(72) Inventors: Igor B. RONINSON, Lexington, SC (US); Eugenia V. BROUDE, Lexington, SC (US); Mengqian CHEN, Lexington, SC (US)

(21) Appl. No.: 18/552,395

(22) PCT Filed: Mar. 25, 2022
(86) PCT No.: PCT/US2022/021983

§ 371 Date: Sep. 25, 2023

Related U.S. Application Data
(60) Provisional application No. 63/165,877, filed on Mar. 25, 2021.

(51) Int. Cl. A61K 31/551; A61K 31/4725; A61P 29/00; A61P 37/06

(52) U.S. Cl. CPC A61K 31/551; A61K 31/4725; A61P 29/00; A61P 37/06

(57) ABSTRACT

Disclosed herein are methods for treating a subject comprising the administration of an effective amount of an inhibitor of CDK8 and CDK19 to a subject in need of a treatment for a cytokine storm or elevated amounts of a multiplicity of different cytokine-storm mediating cytokines.

### CROSS REFERENCE TO RELATED APPLICATIONS

This application claims benefit of priority to U.S. patent application Ser. No. 63/165,877, filed Mar. 25, 2021, the contents of which are incorporated by reference in their entirety.

### STATEMENT REGARDING FEDERALLY SPONSORED RESEARCH

This invention was made with government support under P20 GM109091 and R44 CA203184 awarded by the National Institutes of Health. The government has certain rights in the invention.

### BACKGROUND OF THE INVENTION

Cytokine storm, a.k.a. cytokine release syndrome (CRS), a life-threatening hyperinflammatory response involving elevated amounts of circulating cytokines and immune-cell hyperactivation, can be triggered by bacterial and viral pathogens, cancers, autoimmune conditions, and certain immunotherapies. Its signature feature is massive overproduction of multiple pro-inflammatory cytokines by different cells. The elevated circulating cytokine amounts are associated with acute systemic inflammatory symptoms and dysfunction of secondary organs (often renal, hepatic, or pulmonary) due to inflammation that may lead to death.

Much effort is being devoted to cytokine storm suppression. The understanding that the cytokine storm may contribute to COVID-19 severity and mortality has intensified these efforts. Most drugs that are intended to minimize the cytokine storm that are currently in clinical trials are monoclonal antibodies that act only on a single target, such as an individual cytokine or cytokine receptor. Examples of such drugs include anti-IL-6-receptor antibodies tocilizumab and sarilumab, anti-IL-6 antibody siltuximab (1) and an anti-GM-CSF monoclonal antibody lenzilumab (2). Inhibition of signal transduction pathways associated with cytokine storm, such as NFκB, JAK-STAT, mTOR and MAPK, may have a broader effect against the induction of multiple cytokines. For example, the JAK1/2 inhibitor, baricitinib, showed a general effect against the SARS-COV-2-induced expression of multiple cytokines (3). On the other hand, global inhibition of signal transduction pathways, in particular NFκB, the principal cytokine-inducing transcription factor, leads to systemic toxicity and also suppresses innate immunity (4).

CDK8/19 inhibitors may suppress the induction of transcription of some genes by NFκB (US 2014/0309224). CDK8 (ubiquitously expressed) and CDK19 (expressed in some cell types) are two isoforms of Mediator kinase, the enzymatic component of the CDK module that binds to the transcriptional Mediator protein complex. In addition to CDK8/19, the CDK module also includes Cyclin C, MED12 and MED13 (5). In contrast to other transcriptional CDKs, such as CDK7 or CDK9, CDK8/19 are not a part of the overall transcription machinery (5) but act as cofactors or modifiers of several transcription factors, including STATs (6), β-catenin/TCF/LEF (7), SMADs (8, 9), MYC (10), Notch (11), HIFla (12), AP1 (13), ER (14) and NFκB (15). CDK8/19 directly phosphorylate some transcription factors (SMADs, STATs, AP1, Notch) and mediate C-terminal domain phosphorylation of RNA polymerase II (required for completing gene transcription), in the specific context of newly induced genes (12, 15, 16). CDK8/19 inhibition has a unique transcriptional effect: it impacts primarily de novo-induced but not basal transcription (14, 15), defining CDK8/19 Mediator kinase as a regulator of transcriptional reprogramming (5, 15, 17). CDK8/19 are required for embryonic development, a process driven by transcriptional reprogramming (18, 19), but CDK8 knockout has no phenotypic effects in adult animals (10). Although systemic toxicity was reported for two Mediator kinase inhibitors (20), this toxicity was later shown to be due to off-target effects of these compounds (21). While NFκB plays a key role in the induction of transcription of many cytokines, there is no evidence that NFκB inhibition can suppress the multifactorial network responsible for the induction of numerous cytokines in the context of cytokine storm. Further analysis on the effects of CDK8/19 inhibitors on NFκβ-induced gene expression in different cell lines revealed that CDK8/19 inhibition suppresses the induction of some, but not all, NFκβ-inducible genes, and that the impact of CDK8/19 inhibitors on NFκβ-induced gene expression varies among cell lines (15). NFκβ-inducible genes that most commonly responded to CDK8/19 inhibition in solid tumor cells encode cytokines CXCL1, CXCL2 and IL-8 (15), but not the major mediators of cytokine storm, such as IL-6 or TNFα.

The role of CDK8 and CDK19 in the expression of inflammatory cytokines is unclear. Yamamota et al. (22) investigated the role of CDK8 and CDK 19 in the expression of inflammatory cytokines in RPMI8226 human myeloma cell line by using toll-like receptor 9 (TLR9) agonist ODN2006 to induce inflammatory gene expression. TLR9 stimulation with ODN2006 upregulated the expression of IL-8, IL-10, PTX3, CCL2, CCL3 and CCL4 but not of IL-6, TNF, CXCR4 or CXCL2. shRNA knockdown of CDK8 and CDK19 moderately inhibited TLR9-induced expression of IL-8 and CCL2, while providing a stronger inhibition of PTX3 and IL-10 ((22)). Given that there was no effect on IL-6 and TNF-α, two of the key mediators of cytokine storm in this system, and the fact that IL-10, while upregulated in cytokine storm, has primarily an anti-inflammatory function, this study did not suggest that pharmacological CDK8/19 inhibitors would be capable of suppressing cytokine storm. Although Yamamoto et. al. (22) found IL-10 to be downregulated by CDK8/19 knockdown, Johannessen et al. (13) observed the opposite: different CDK8/19 inhibitors strongly induced IL-10 production during innate immune activation. Concurrently with the induction of the anti-inflammatory IL-10, Johannessen et al. (13) observed moderate (˜2-fold) inhibition of IL-6 (the only tested pro-inflammatory cytokine in that paper) in murine bone marrow derived dendritic cells and macrophages, upon treatment with CDK8/19 inhibitors. Based on the IL-10 induction, Johannessen et al. (13) proposed that CDK8/19 inhibitors should have anti-inflammatory activity. However, the proposed anti-inflammatory effect of CDK8/19 inhibitors was not tested by Johannessen et al. (13) in vivo or in human immune cells. As a result, there is a need in the art for compositions and methods for treating cytokine storm or elevated amount of a multiplicity of different cytokine-storm mediating cytokines.

### BRIEF SUMMARY OF THE INVENTION

Disclosed herein are methods for treating a subject comprising the administration of an effective amount of an inhibitor of CDK8 and CDK19 to a subject in need of a treatment for a cytokine storm or elevated amounts of a multiplicity of different cytokine-storm mediating cytokines. In some embodiments, elevated amounts of a multiplicity of different cytokines in the subject are induced by a pathogen, such as a viral or bacterial pathogen, including SARS-COV-2, a cancer, an autoimmune condition, or an immunotherapy. In some embodiments, the subject is in need of a treatment for acute respiratory distress syndrome, hypoxemia, acute systemic inflammation, secondary organ dysfunction, or any combination thereof. The effective amount of the inhibitor of CDK8 and CDK19 may be administered prior to or after induction of elevated amounts of a multiplicity of different cytokines in the subject. Suitably, the inhibitor of CDK8 and CDK 19 may be administered to a human subject. An exemplary inhibitor of CDK8 and CDK19 for use in the methods described herein is 3-amino-4-(4-(4 (dimethylcarbamoyl) phenyl)-1,4-diazepan-1-yl)thieno[2,3-b]pyridine-2-carboxamide (15u) or 2-(4-(4-(isoquinolin-4-yl)phenyl)-1H-pyrazol-1-yl)-N,N-dimethylacetamide (BI-1347).

In some embodiments, the effective amount of the inhibitor of CDK8 and CDK 19 reduces the amount of a multiplicity of different cytokine-storm mediating cytokines or RNA expression of a multiplicity of different cytokine-storm mediating cytokines. In some embodiments, the amount of two, three, four, or more of IL-6, TNFα, GM-CSF, IFN-γ, IL-1α, IL-1β, IL-8, IL-12 (p40), IL-12 (p70), IL-18, MIG/CXCL9, MIP−1α, MIP-1β, and TNFβ is reduced.

In some embodiments, the effective amount of the inhibitor of CDK8 and CDK19 does not significantly reduce the amount of an anti-inflammatory cytokine. In particular embodiments, the effective amount of the inhibitor of CDK8 and CDK19 does not significantly reduce the amount of IL-10.

Another aspect of the invention provides for a method for treating a subject in need of a treatment for a cytokine storm or elevated amounts of a multiplicity of different cytokine-storm mediating cytokines, the method comprising detecting two or more different cytokine-storm mediating cytokines or RNA expression thereof in a sample obtained from the subject and administering an effective amount of an inhibitor of CDK8 and CDK 19 to the subject if the subject has elevated amounts of the two or more different cytokine-storm mediating cytokines or RNA expression thereof.

Another aspect of the invention provides for a method for identifying patients in need a treatment for a cytokine storm or elevated amounts of a multiplicity of different cytokine-storm mediating cytokines, the method comprising detecting for elevated amounts of two or more different cytokine-storm mediating cytokines or RNA expression thereof in a sample from the subject, wherein the subject is eligible for treatment with an effective amount of an inhibitor of CDK8 and CDK 19 if the subject has elevated amounts of the two or more different cytokine-storm mediating cytokines or RNA expression thereof.

These and other aspects of the invention will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Effect of LPS and 15u (SNX) treatment on the expression of indicated cytokines (pg/mL) in male C57BL/6 mice before LPS treatment (pre) and 2 hrs or 6 hrs after LPS dosing.

FIG. 2. Effect of LPS and 15u treatment on the expression of indicated human cytokines (pg/mL) in plasma samples of humanized Hu-NoG-EXL mice (in order of LPS fold induction). 33 of 48 human cytokines were induced by LPS>2-fold. G1_pre: before LPS treatment; G1_6h: 6 hrs after LPS treatment; G2_6 h: 7 hrs after 15u and 6 hrs after LPS.

FIG. 3. Effect of LPS and 15u treatment on the expression of indicated mouse cytokines (pg/mL) in plasma samples of humanized Hu-NoG-EXL mice (in order of LPS fold induction). 27 of 32 mouse cytokines were induced by LPS>2-fold. G1_pre: before LPS treatment; G1_6 h: 6 hrs after LPS treatment; G2_6 h: 7 hrs after 15u and 6 hrs after LPS.

FIGS. 4A-4B. RNA Expression of human (FIG. 4A) and mouse cytokines (FIG. 4B) (fpkm), associated with cytokine storm, in spleens of humanized Hu-NoG-EXL mice 6 hrs after LPS treatment. G1: LPS only; G2: 15u before LPS; G3: 15u after LPS.

FIG. 5. Effect of treatment with LPS and with 15u (SNX), administered alone or 1 hr before or 0.5 hr after LPS, and with BI-1347 (BI) administered 1 hr before LPS, on the expression of indicated human cytokines (pg/mL) in plasma samples of humanized Hu-NoG-EXL mice. Ctrl: no treatment; SNX: 15u alone; LPS: LPS alone; SNX+LPS: 15u before LPS; LPS+SNX: 15u after LPS; BI+LPS: BI-1347 before LPS.

### DETAILED DESCRIPTION OF THE INVENTION

Disclosed herein are methods for treating cytokine storm with inhibitors of CDK8 and CDK19 (CDK8/19 inhibitors). As demonstrated in the Examples, inhibitors of CDK8 and CDK19 are effective for reducing the amount of protein or RNA expression of a multiplicity of different cytokine-storm associated cytokines. Notably, however, inhibitors of CDK8 and CDK19 do not reduce the level of anti-inflammatory cytokines, such as IL-10. The demonstrated effect against a multiplicity of pro-inflammatory cytokines but not the anti-inflammatory IL-10 provides for a surprising advantage of CDK8/19 inhibitors for treating cytokine storms or resultant symptoms.

Methods for treating subjects with the compounds disclosed herein are provided. Suitably, the methods for treating a subject comprise administering to the subject an effective amount of one or more inhibitors of CDK8 and CDK 19 or a pharmaceutical composition comprising the effective amount of one or more inhibitors of CDK8 and CDK19. As used herein, a “subject” may be interchangeable with “patient” or “individual” and means an animal, which may be a human or non-human animal, in need of treatment. In particular embodiments, the subject is a human subject.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. In some embodiments, the subject is responsive to therapy with one or more of the compounds disclosed herein in combination with one or more additional therapeutic agents.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be reducing the amount of a multiplicity of different cytokine storm mediating cytokines.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A “subject in need of treatment” may include a subject having a disease, disorder, or condition that may be characterized as a cytokine storm. Cytokine storm, a.k.a. cytokine release syndrome (CRS), is a life-threatening hyperinflammatory response involving elevated amounts of circulating cytokines and immune-cell hyperactivation. A cytokine storm can be triggered by pathogens (including viral and bacterial pathogens), cancers, autoimmune conditions, and certain immunotherapies. Cytokine storm may cause acute respiratory distress syndrome (ARDS). Cytokine storm is an umbrella term encompassing several disorders of immune dysregulation characterized by elevated amounts of circulating cytokines, acute systemic inflammation, and secondary organ dysfunction. Multi-organ failure may occur if inadequately treated. Organs and systems affected by a cytokine storm may include, lungs, liver, kidneys, heart, skin, vascular system, lymphatic system, nervous system, rheumatologic system, gastrointestinal system, or any combination thereof. Although the initial drivers may differ, late-stage clinical manifestations of cytokine storm converge and often overlap. Nearly all patients with cytokine storm are febrile, and the fever may be high grade in severe cases. In addition, patients may have fatigue, anorexia, headache, rash, diarrhea, arthralgia, myalgia, and neuropsychiatric findings.

Cytokine induction is mediated by several signaling pathways, including NFκB, JAK-STAT, mTOR and MAPK. The elevated circulating cytokine amounts are associated with acute systemic inflammatory symptoms and dysfunction of secondary organs (often renal, hepatic, or pulmonary) due to inflammation. Many patients have cough and other respiratory symptoms that can progress to acute respiratory distress syndrome (ARDS), with hypoxemia that may require mechanical ventilation. Cytokine storm has been implicated in the severity and mortality of diverse bacterial diseases causing sepsis, viral diseases such as influenza and COVID-19, hemophagocytic lymphohistiocytosis (HLH), autoinflammatory disorders, autoimmune disorders, and immunotherapies such as Coley's toxins, T-cell therapy, or CAR-T therapy.

These symptoms may be due directly to cytokine-induced tissue damage or acute-phase physiological changes or may result from immune cell-mediated responses. A signature feature of a cytokine storm is overproduction of a multiplicity of pro-inflammatory cytokines, such as interleukin (IL)-1β, IL-6, IL-18, tumor necrosis factor (TNF), interferon (IFN)-γ, GM-CSF, MIP-la and others. Cytokines are a category of small proteins, typically, 5-20 kDa, important in cell signaling and are immunomodulating agents. Cytokines include chemokines, interferons, interleukins, lymphokines, and tumor necrosis factors. These mediators of cytokine storm are produced by and stimulate different types of immune cells; a network of interactions between different cytokines and immune cells leads to continuous high cytokine amounts in the body. Table 1 lists cytokines that are identified as mediating a cytokine storm (Fajgenbaum and June, 2020).

TABLE 1
Mediators of cytokine storm.

| Cytokines and | Gene | Cytokines and | Gene |
|---|---|---|---|
| chemokines | names | chemokines | names |
| Interleukin-1 | IL1A | Interferon-γ | IFNG |
| Interleukin-1 | IL1B | Tumor necrosis factor (alpha) | TNF |
| Interleukin-2 | IL2 | Tumor necrosis factor (beta) | LTA |
| Interleukin-6 | IL6 | GM-CSF | CSF2 |
| Interleukin-9 | IL9 | VEGF-A | VEGFA |
| Interleukin-10 | IL10 | VEGF-B | VEGFB |
| Interleukin-12 | IL12A | VEGF-C | VEGFC |
| Interleukin-12 | IL12B | VEGF-D | VEGFD |
| Interleukin-17 | IL17A | Interleukin-8 (CXCL8) | CXCL8 |
| Interleukin-17 | IL17B | MIG (CXCL9) | CXCL9 |
| Interleukin-17 | IL17C | IP-10 (CXCL10) | CXCL10 |
| Interleukin-17 | IL17D | MCP-1 (CCL2) | CCL2 |
| Interleukin-17 | IL17F | MIP-1α (CCL3) | CCL3 |
| Interleukin-18 | IL18 | MIP-1β (CCL4) | CCL4 |
| Interleukin-33 | IL33 | BLC (CXCL13) | CXCL13 |

As used herein, an “elevated amount” means an amount above the mean of the particular cytokine or substance found in a representative population that is not in need of a treatment. In some embodiments, the elevated amount may be a statistically significant amount or one, two, or three standard deviations above the mean. The methods described herein may be performed after induction of the elevated amounts of a multiplicity of different cytokines in the subject. Alternatively, the methods described herein may be performed prior to induction of the elevated amounts of a multiplicity of different cytokines in the subject to reduce the severity or duration of elevated amounts of a multiplicity of different cytokines in the subject.

The presence of an elevated amount of cytokine-storm mediated cytokines in a sample obtained from a subject can be used to identify whether the subject can benefit from administration of an inhibitor of CDK8 and CDK19. Samples may be obtained from a subject and evaluated for elevated amounts of a multiplicity of different cytokine-storm mediating cytokines. As used herein, “sample” includes any bodily fluid or tissue obtained from a subject useful for determining the presence or amount of one or more cytokine-storm mediating cytokines. Examples of samples include blood, plasma, serum, saliva, urine, or other bodily fluids. In some embodiments, different samples may be obtained from the subject to determine the presence of absence of one or more different cytokine-storm mediating cytokines. In some embodiments, the amount of cytokine-storm mediating cytokines are directly measured. In other embodiments, the amount of cytokine-storm mediating cytokines are indirectly measured, such as through measurement of the amount of RNA expression for a particular cytokine. Those skilled in the art can detect the presence or amount of cytokines, such as through a Human Cytokine 48-Plex Discovery Assay as disclosed in the Examples. Where cytokine-storm mediating cytokines are found to be elevated in the subject, the subject may be administered inhibitors of CDK8 and CDK19.

As used herein, “multiplicity” means two or more cytokines or two or more things depending on context. In some embodiments, multiplicity means 2, 3, 4, 5, 6, 7, 8, 9, 10, 11, 12, 13, 14, 15, 16, 17, 18, 19, 20, or more cytokines or things. A multiplicity of different cytokine-storm mediating cytokines may include any two or more of the cytokines listed in Table 1. In some embodiments, a multiplicity of different cytokine-storm mediating cytokines may be selected from any two or more of IL-6, TNFα, GM-CSF, IFN-γ, IL-1α, IL-1β, IL−8, IL-12 (p40), IL-12 (p70), IL-18, MIG/CXCL9, MIP-1α, MIP-1β, and TNFβ.

The methods described herein provide for a reduction in the amount of one or more cytokines. As used herein, “reduces the amount of a cytokine” means to reduce the amount of a cytokine by a statistically significant amount or by at least 20% and “reduces the amount of a multiplicity of different cytokines” means to reduce the amount of two or more cytokines a statistically significant amount or by at least 20% in the subject. In some embodiments, the reduction may be at least 30%, 40%, 50%, 60%, 70%, 80%, or more.

The methods described herein provide for a reduction in the amount of RNA expression of one or more cytokines in a sample from a treated subject. As used herein, “reduces RNA expression” means to reduce the amount of an RNA encoding a cytokine by a statistically significant amount or by at least 20% and “reduces RNA expression of a multiplicity of different cytokines” means to reduce the amount of RNA encoding two or more cytokines by a statistically significant amount or by at least 20%. In some embodiments, the reduction may be at least 30%, 40%, 50%, 60%, 70%, 80%, or more.

In some embodiments, a multiplicity of different samples may be obtained from the subject at different time points to monitor the subject. In some embodiments, a sample is obtained from the subject prior to administration of the inhibitor of CDK8 and CDK 19 and one or more additional samples are obtained after administration of the inhibitor of CDK8 and CDK19. The amount of cytokine-storm mediating cytokines directly or indirectly detected at different time points can be used to monitor reduction in the amount of cytokine-storm mediating cytokines. Such information may be used to determine when to stop administration of the inhibitor of CDK8 and CDK19.

An advantage of the presently disclosed technology is that it may reduce the amount a multiplicity of different cytokine-storm mediating cytokines but not anti-inflammatory cytokines, such as IL-10 in a treated subject. Anti-inflammatory cytokines are important for antagonizing inflammatory-cell populations and preventing hyperactivity of the immune response. Numerous regulatory cytokines such as IL-10 and natural cytokine antagonists such as IL-IRA serve as buffers to limit systemic off-target effects. IL-10 inhibits the production of TNF, IL-1, IL-6, and IL-12 and down-regulates antigen presentation. Furthermore, in mice lacking interleukin-10, infection leads to cytokine storm (Fajgenbaum and June, 2020). Though IL-10 and IL-IRA are often elevated in cytokine storm, this finding most likely reflects a secondary, albeit insufficient, counter regulatory response to the pro-inflammatory cytokines. Accordingly, CDK8/19 inhibitors can reduce the levels of cytokine-storm mediating cytokines without a significant reduction in anti-inflammatory cytokines. As used herein, “significantly reduce” means a reduction that is statistically significant or where the reduction is at least 20%.

The Examples presented herein demonstrate the utility of the inhibitors of transcription-regulating kinases CDK8 and CDK 19, such as described in (23-25), for the suppression of cytokine storm. 3-amino-4-(4-(4 (dimethylcarbamoyl) phenyl)-1,4-diazepan-1-yl)thieno[2,3-b]pyridine-2-carboxamide (15u) and 2-(4-(4-(isoquinolin-4-yl)phenyl)-1H-pyrazol-1-yl)-N,N-dimethylacetamide (BI-1347), disclosed in WO 2017/202719, were used in the Examples but other inhibitors of CDK8 and CDK19 may also be used in the presently disclosed methods, including selective CDK8/19 inhibitors disclosed in U.S. Pat. Nos. 8,598,344, 9,321,737, 9,409,873; US 2020/0062728, WO 2017/202719; WO 2019/168446; WO 2020/160537; WO 2020/237014, (24, 26, 27); (28-34), the contents of each is incorporated by reference in their entirety.

As used herein, an inhibitor that “selectively inhibits CDK8 and CDK19” is a compound that inhibits CDK8 and CDK19 without inhibiting the majority of other kinases. Selective inhibition can be determined by kinome profiling using an active site-directed competition binding assay to quantitatively measure interactions between the compound and a plurality of human kinases and disease relevant mutant variants. In some embodiments, the inhibitor that selectively inhibits CDK8 and CDK 19 has an S-score of S(35)<0.1 or S(10)<0.01 at an effective amount of the CDK8 and CDK19 inhibitor, where S(#)=(number of non-mutant kinases with % Ctrl (or POC)<#)/(number of non-mutant kinases tested). In some embodiments, the inhibitor that selectively inhibits CDK8 and CDK 19 has an S-score of S(35)<0.08, 0.06, 0.04, or 0.02. In some embodiments, the inhibitor that selectively inhibits CDK8 and CDK19 has an S-score of S(10)<0.080, 0.006, or 0.004. For example, 15u has a S(35) and S(10) against a panel of 468 kinases of less than 0.02 and 0.004, respectively, at 2000 nM (WO 2020/237014).

The CDK8/19 inhibitors disclosed herein may be formulated as pharmaceutical compositions that include: an effective amount of one or more compounds and one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods disclosed herein may be administered as a single compound or a combination of compounds.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Pharmaceutically acceptable esters and amides of the compounds can also be employed in the compositions and methods disclosed herein.

In addition, the methods disclosed herein may be practiced using solvate forms of the compounds or salts, esters, and/or amides, thereof. Solvate forms may include ethanol solvates, hydrates, and the like.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The ability of CDK8/19 inhibitors to suppress the induction of multiple cytokines in vivo is demonstrated using a commonly used trigger of cytokine storm, bacterial lipopolysaccharide (LPS) (35, 36). LPS (endotoxin) binds the CD14/TLR4/MD2 receptor complex in many cell types, but especially in monocytes, dendritic cells, macrophages and B cells, triggering the secretion of pro-inflammatory cytokines (37). As a selective CDK8/19 inhibitor, we have used 15u (a.k.a. SNX), a potent, selective and metabolically stable CDK8/19 kinase inhibitor (patent applications PCT/US2020/016394; PCT/US2020/033937). Surprisingly, we have discovered that CDK8/19 inhibitor did not suppress LPS-induced cytokine induction in mice. However, when tested in “humanized” mice, reconstituted with human CD34-positive hematopoietic stem cells, CDK8/19 inhibition strongly and broadly suppressed the induction of almost all the human cytokines associated with the cytokine storm, while having very little effect on LPS-induced mouse cytokines. Another, chemically distinct selective CDK8/19 kinase inhibitor BI-1347 (28), also broadly suppressed the induction of most of the human cytokines. These findings demonstrate that different CDK8/19 inhibitors can be used in humans for the treatment or prevention of cytokine storm, responsible for the severity or mortality of many diseases.

### Example 1. CDK8/19 Inhibitor does not Suppress LPS-Induced Cytokine Expression in C57BL/6 Mice

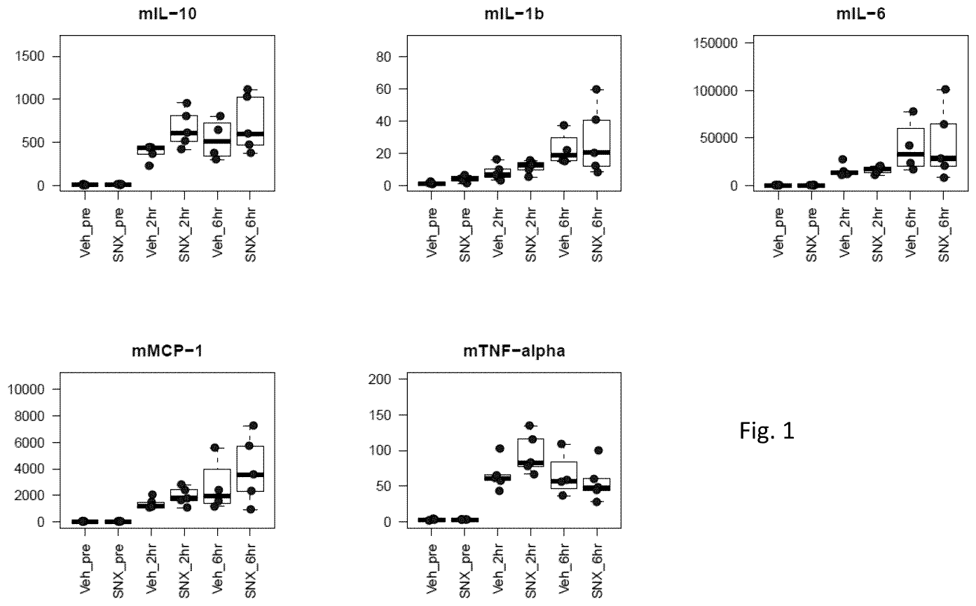

Cytokine storm was induced in C57BL/6 male mice by intraperitoneal (i.p.) injection of LPS (10 mg/kg). Two groups of LPS-treated mice (n=5) received 30 mg/kg 15u dissolved in 30% Propylene Glycol, 70% PEG-400 or vehicle control (5 mice per group), via oral gavage 2 hrs prior to LPS dosing. Blood was collected immediately before LPS dosing, 2 hrs after LPS and 6 hrs after LPS (at which time point the animals were euthanized). Plasma samples (1:100 dilution for 2 hr and 6 hr samples, 1:5 dilution for pre-LPS samples) were used to analyze the cytokines with the MSD U-plex custom panel for the following mouse cytokines: IL-1β, IL-6, IL-10, MCP-1 and TNF-α. The results of the measurements are shown in FIG. 1. 15u treatment had no significant effect on LPS-induced expression of any of the assayed cytokines.

### Example 2. CDK8/19 Inhibitor Suppresses LPS-Induced Expression of Multiple Human Cytokines in Humanized Mice

To test the effect of CDK8/19 inhibition on LPS-induced cytokine expression in human immune cells, we carried out a study on humanized mice transplanted with human hematopoietic stem cells; such mice contain human blood cells of myeloid and lymphoid lineages as well as mouse blood cells. We have used CIEA NOG-EXL mice (Taconic model #13395) engrafted with human umbilical cord blood-derived CD34+ hematopoietic stem cells 21 weeks before the study. All the mice were female, 25-27 weeks old, and had >45% hCD45+ cells in blood. Eight mice were treated with 1 mg/kg LPS i.p.; this dose was selected based on the finding of (38) that 12.5 μg (˜0.5 mg/kg) produced stronger cytokine induction than 50 μg (˜2 mg/kg) in humanized mice. The first group of LPS-treated mice (n=3) (G1) received vehicle alone by oral gavage 1 hr before LPS, the second group (n=3) received 15u at 30 mg/kg by oral gavage 1 hr before LPS, and the third group (n=2) (G3) received 15u at 30 mg/kg by oral gavage 0.5 hrs after LPS. Blood samples were collected from each mouse one week before LPS dosing. Mice were euthanized 6 hrs after LPS dosing and terminal blood samples were collected for cytokine analysis; in addition, spleens were collected for RNA analysis.

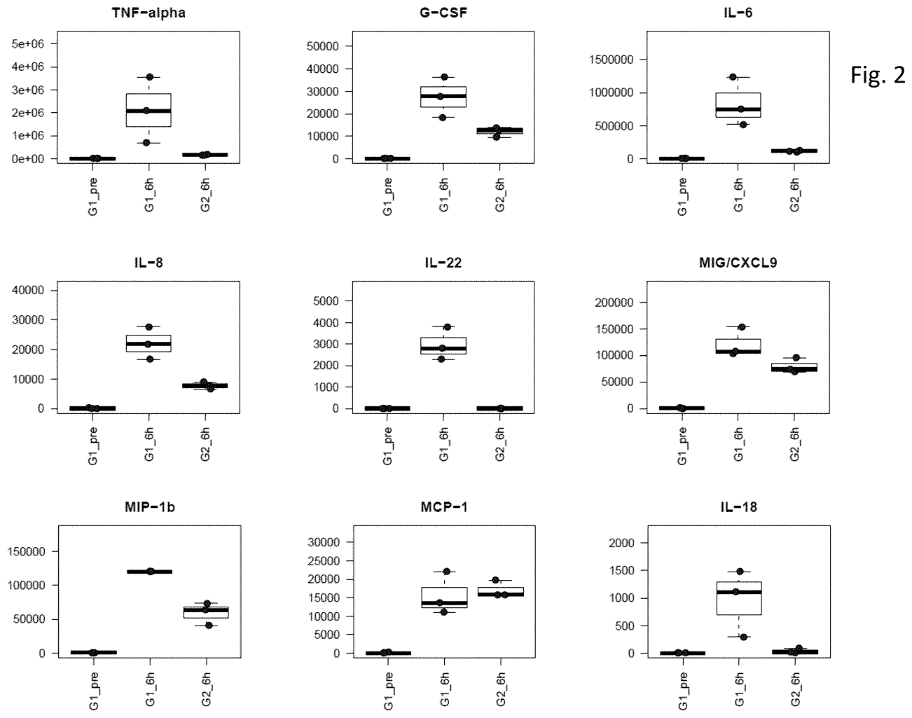

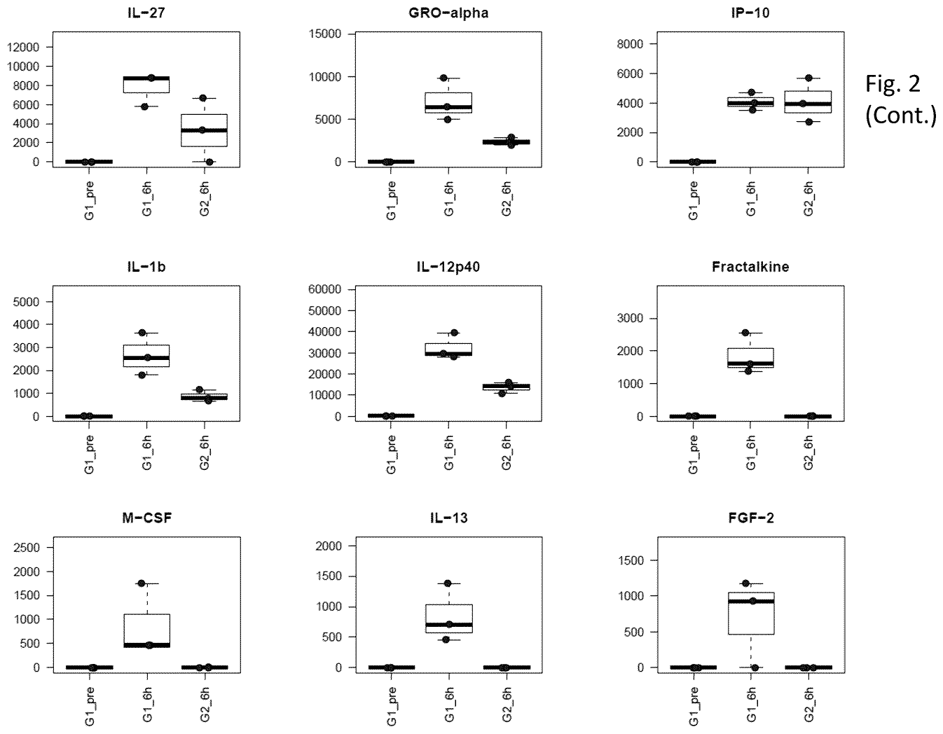

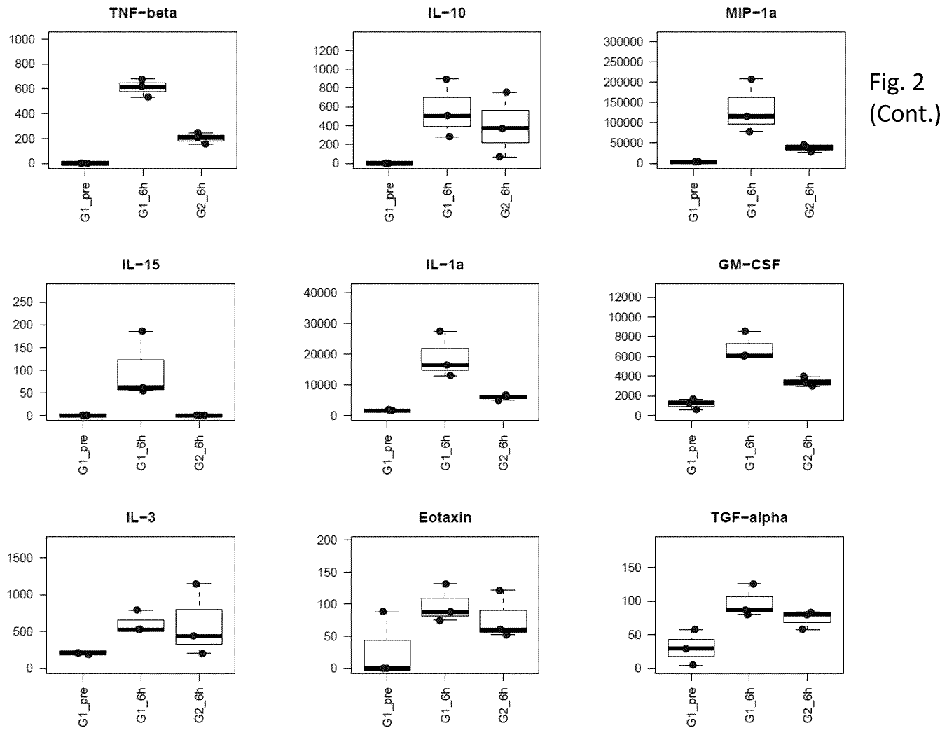

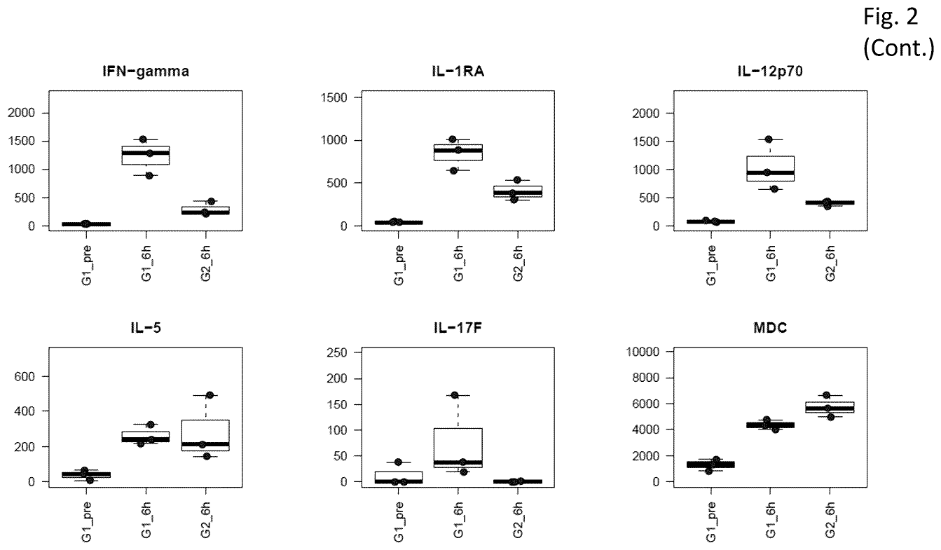

The effects of LPS and 15u on the expression of 48 human cytokines were measured using Human Cytokine 48-Plex Discovery Assay (Eve Technologies) using plasma samples at 1:100 dilution. The assays were done on pre-treated samples from mice of G1 (a pilot assay showed very minor variations in pre-LPS cytokine amounts in different mice), and on post-LPS samples of mice from G1 (LPS alone) and G2 (15u followed by LPS). The results of this analysis are shown in FIG. 2 for 33 of 48 cytokines that were induced by LPS>2-fold (cytokine plots are shown in the order of fold induction by LPS). The induction of 24 of 27 of the most strongly induced cytokines was suppressed by CDK8/19 inhibitor. These included 14 mediators of cytokine storm listed in Table 1: GM-CSF, IFN-γ, IL-1 (α and β), IL-6, IL-8, IL-12 (p40 and p70), IL-18, MIG/CXCL9, MIP-1 (α and β) and TNF (α and β). Only 3 cytokine storm mediators that were induced by LPS were not inhibited by 15u: MCP-1, IP-10 and anti-inflammatory IL-10 (only borderline inhibition of IL-10 was observed). The effect against the majority of pro-inflammatory cytokines but not the anti-inflammatory IL-10 suggests a potential advantage of CDK8/19 inhibitors over JAK inhibitors, such as baricitinib, which inhibits IL-10 signaling and secretion (3).

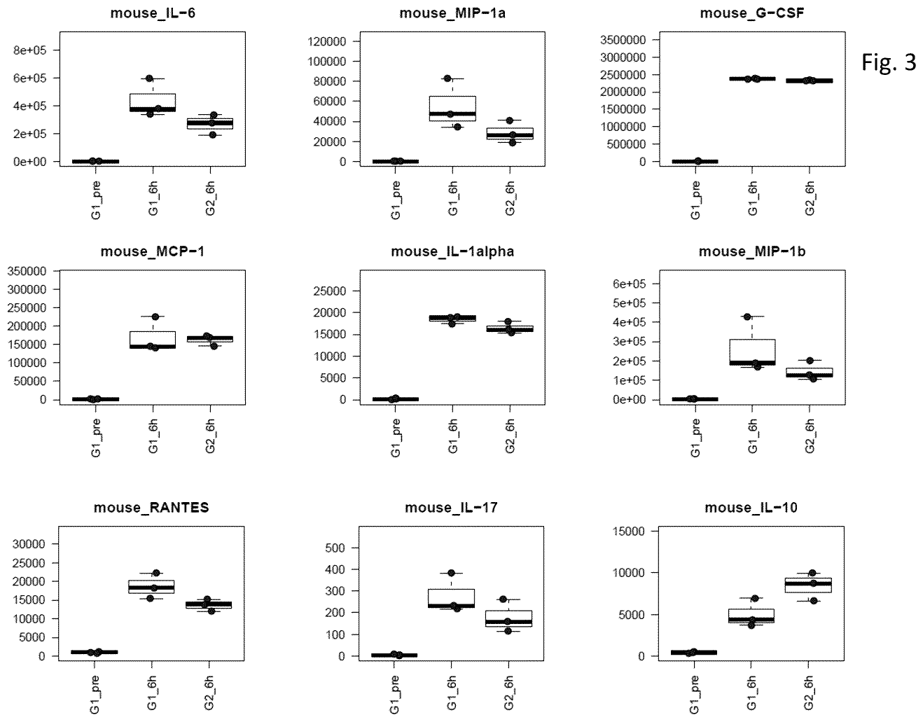

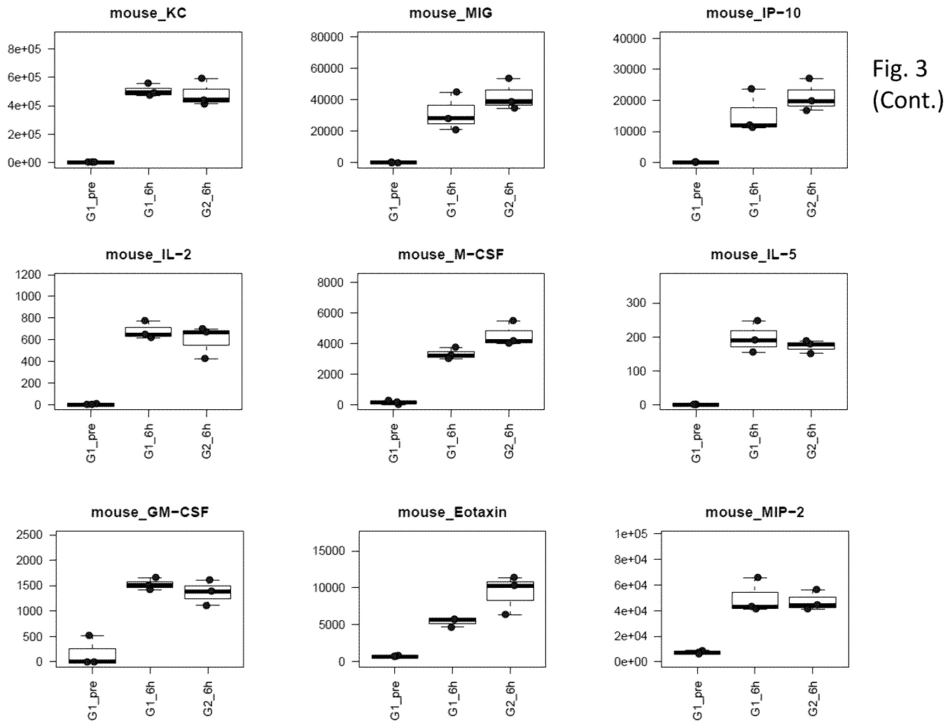

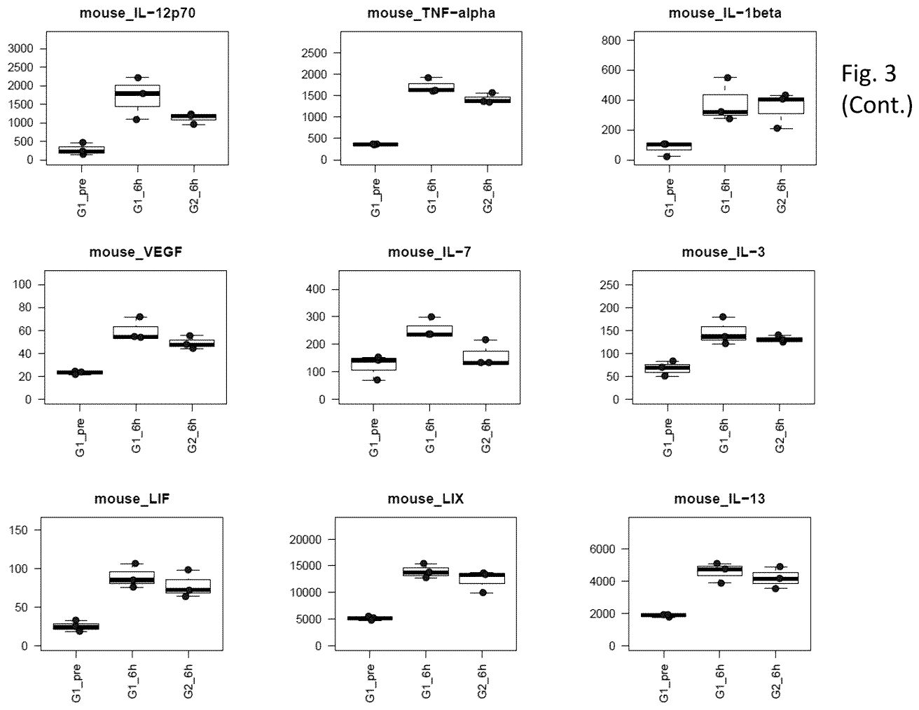

We have also used the same plasma samples (at 1:100 dilution) to measure the expression of 32 mouse cytokines using Mouse Cytokine 32-Plex Discovery Assay (Eve Technologies). The results are shown in FIG. 3. 27 of 32 mouse cytokines were induced by LPS but most of them were unaffected by 15u. The exceptions were the two most strongly LPS-induced cytokines IL-6 and MIP-1A, as well as IL-7, which was weakly inhibited by 15u. Several cytokines appeared to be over-induced by 15u treatment, notably including IL-10 (in contrast to human IL-10 that was not over-induced by 15u, FIG. 2).

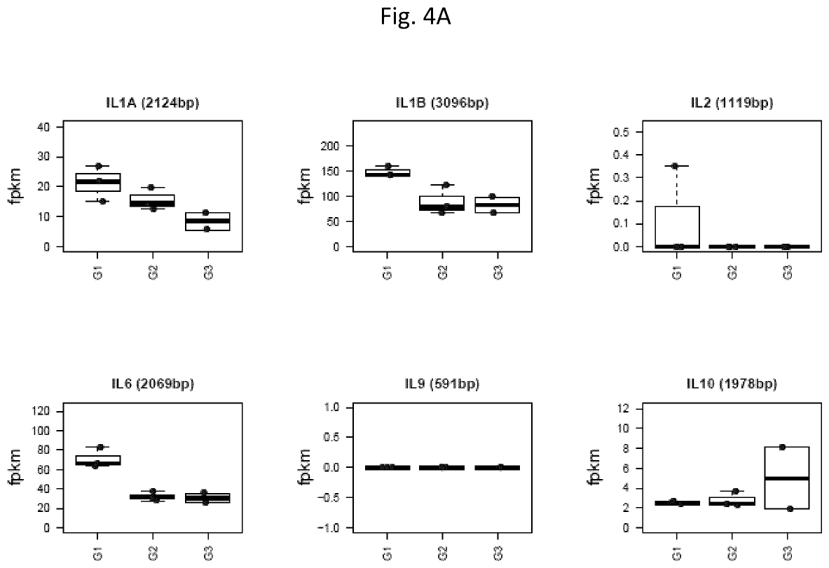

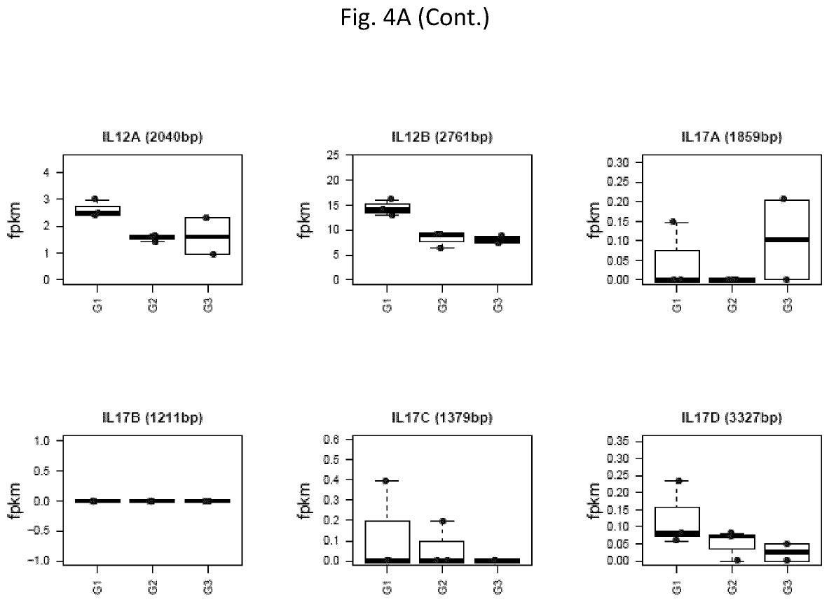

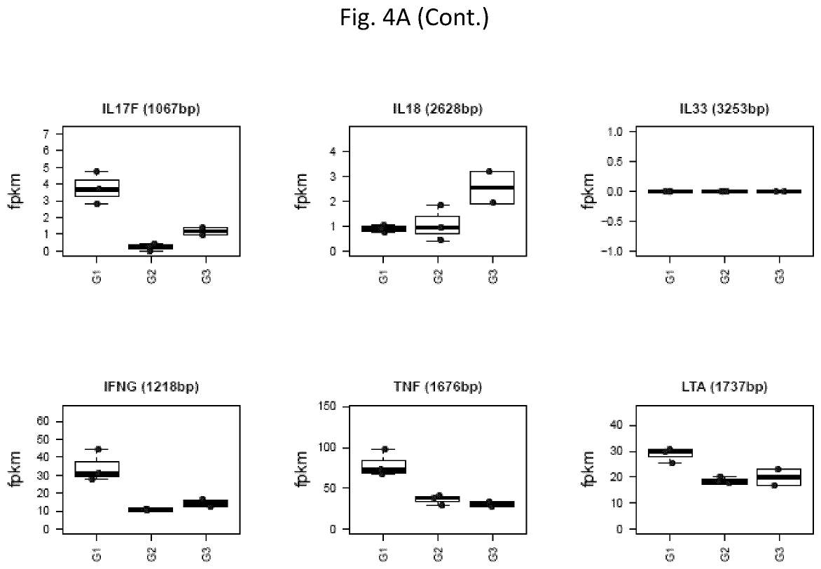

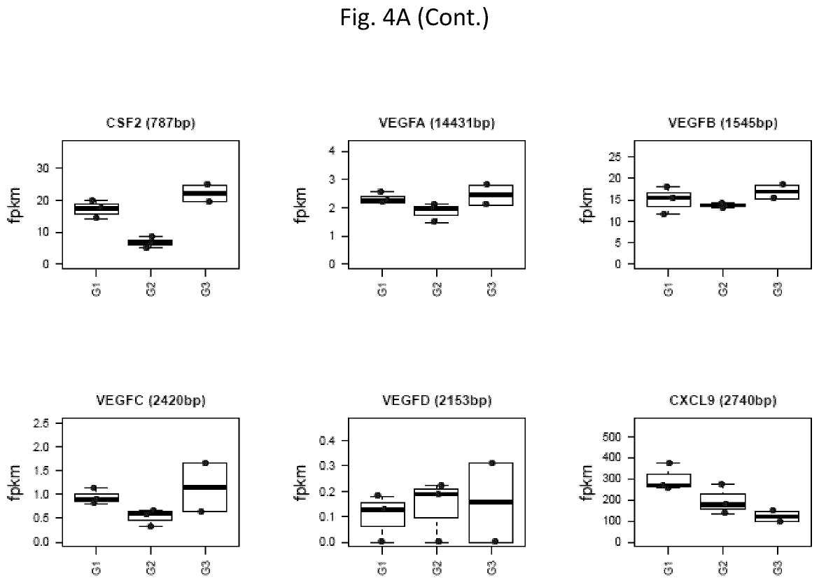

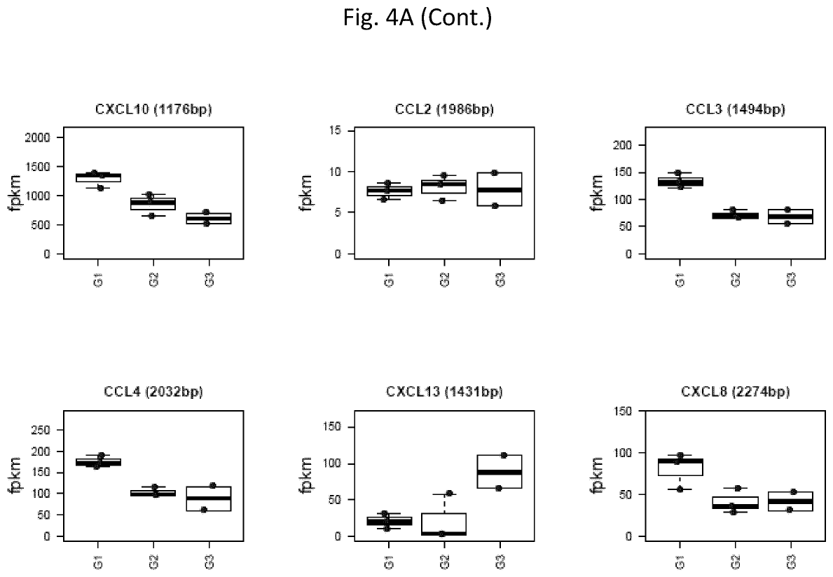

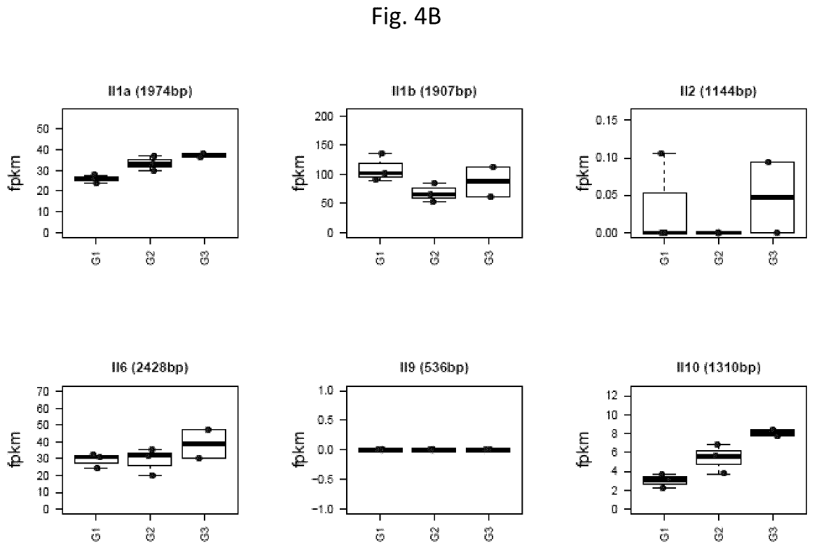

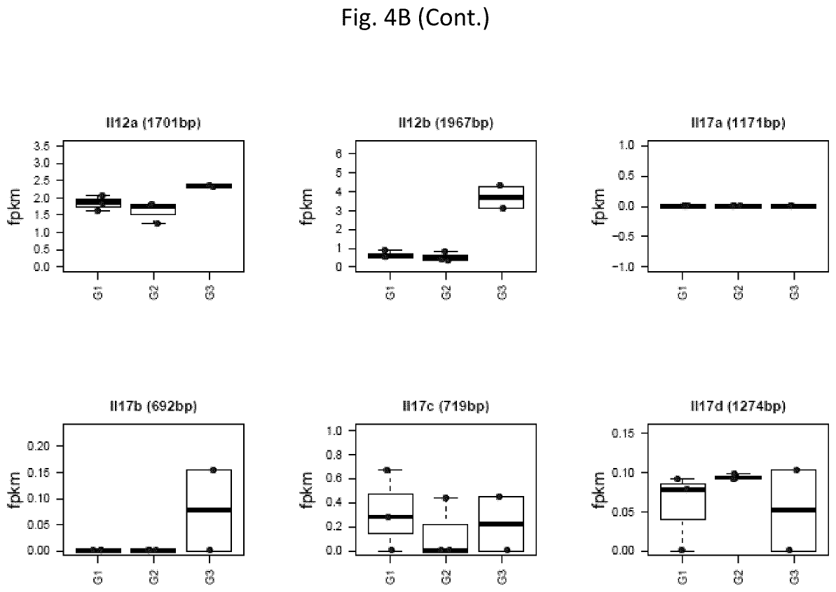

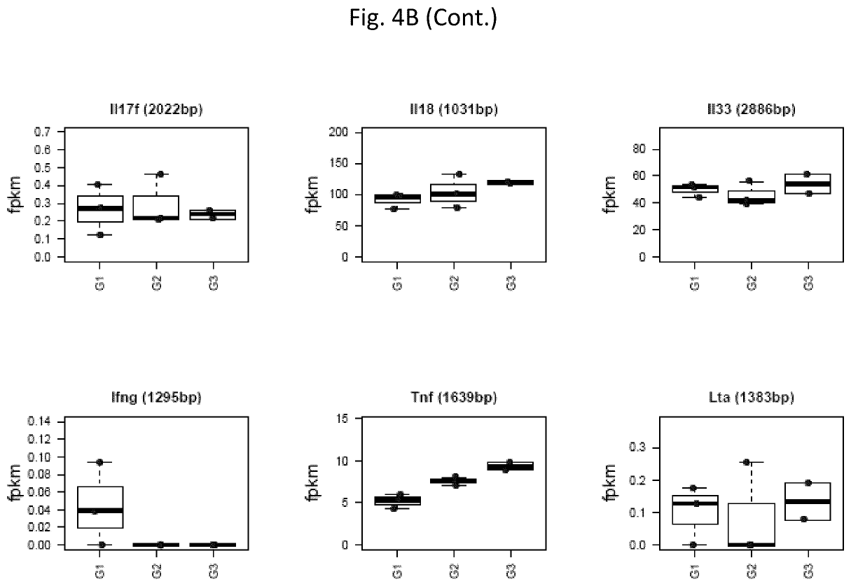

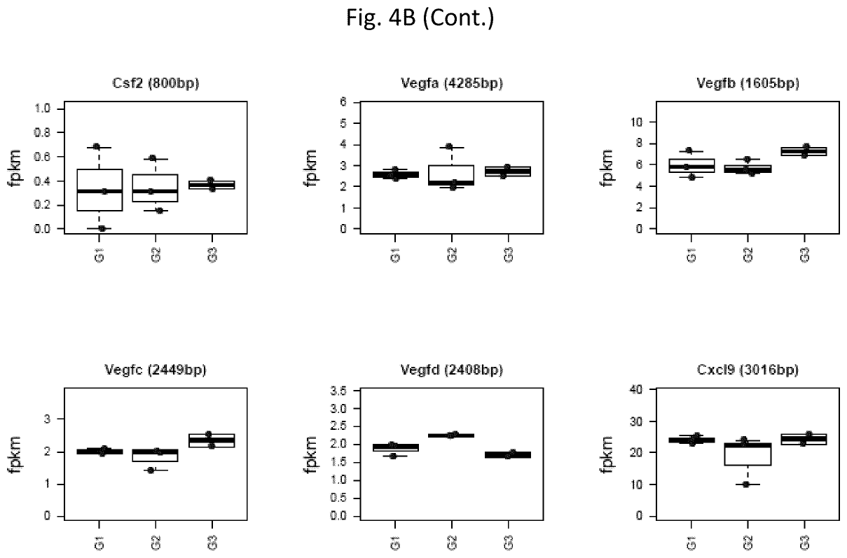

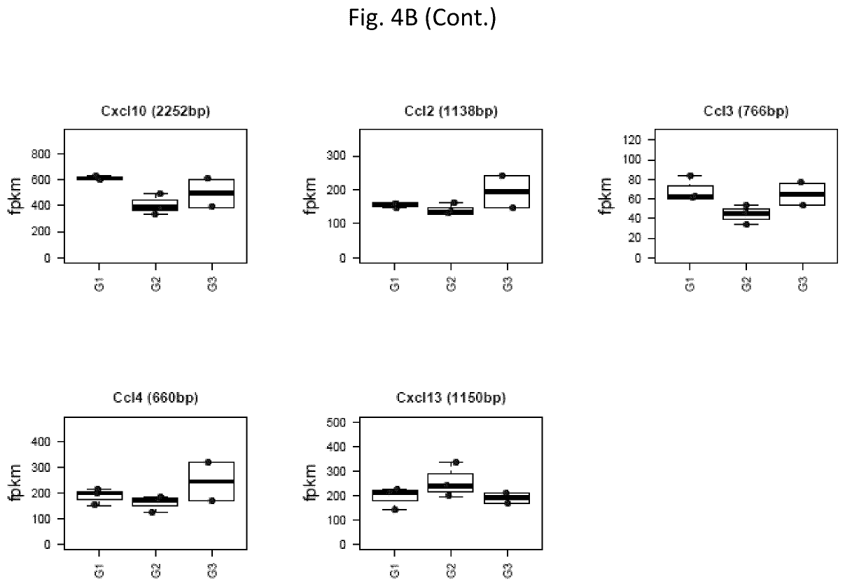

RNA from spleens of mice of G1-3, euthanized 6 hrs after LPS dosing, was analyzed by RNA-Seq. FIGS. 4A-4B shows RNA expression of human and mouse cytokines associated with cytokine storm (Table 1) in spleens of mice treated with LPS alone (G1) or treated with 15u before LPS (G2) or after LPS (G3). 26 of 30 human cytokines and 27 of 29 mouse cytokines were expressed in the spleen. 15u decreased RNA amounts of 18 of 26 human cytokines when administered before LPS and 15 of 26 human cytokines when administered after LPS. In contrast, only 3 of 27 mouse cytokines were decreased in 15u-treated G2 or G3 relative to G1, and 7 mouse cytokines (including TNF and IL-10) were elevated in G2 and especially in G3 relative to G1.

Thus, both protein and RNA analyses show that CDK8/19 inhibitor had a prominent and broad effect on the induction of most of the human cytokines implicated in cytokine storm, whereas mouse cytokines were largely unaffected or only weakly affected. Furthermore, CDK8/19 inhibitor suppresses cytokine storm when administered either before or after the trigger of cytokine induction.

### Example 3. LPS-Induced Expression of Human Cytokines in Humanized Mice is Suppressed by Different CDK8/19 Inhibitors Administered Before or after LPS

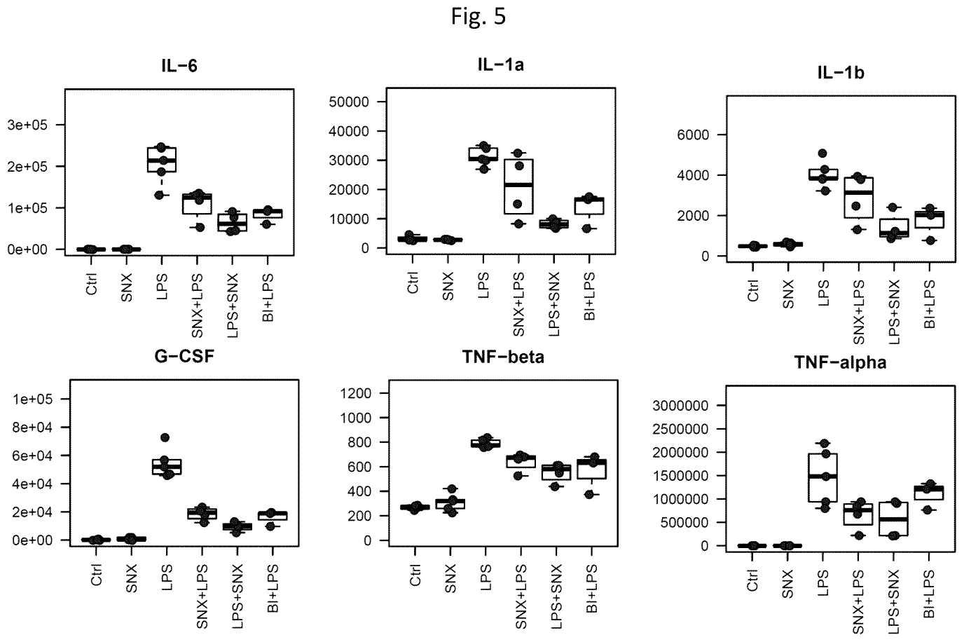

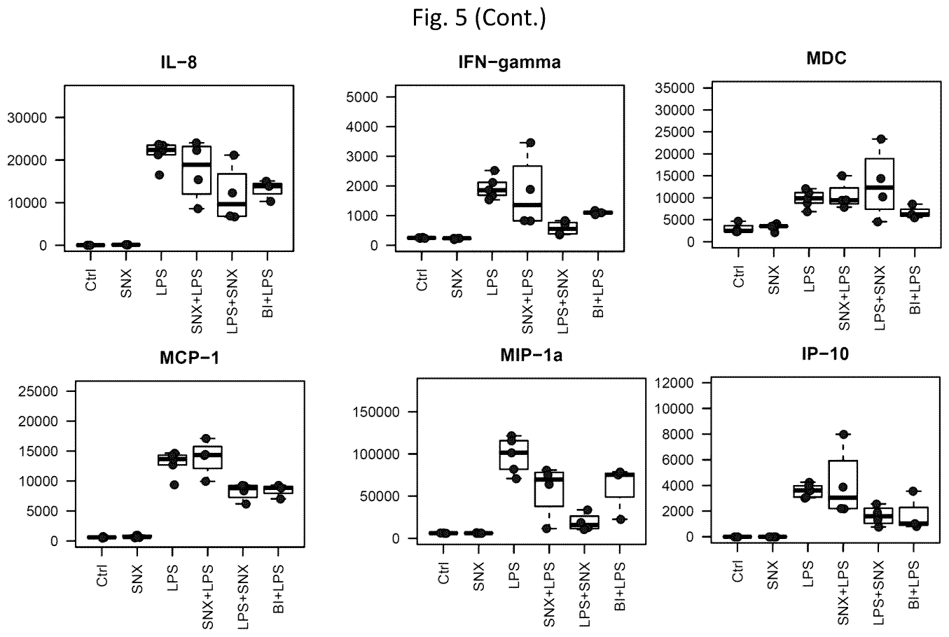

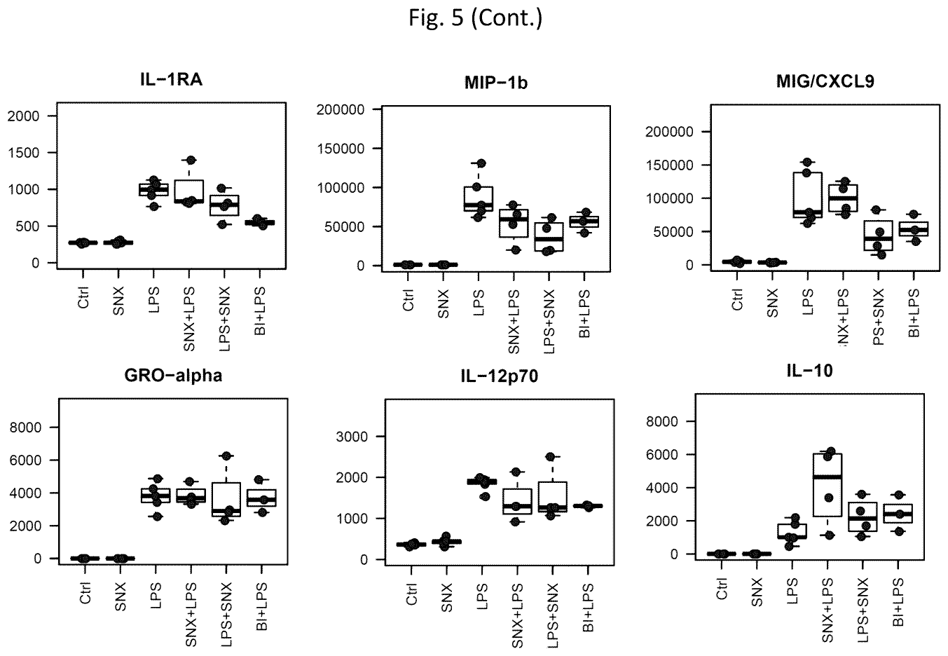

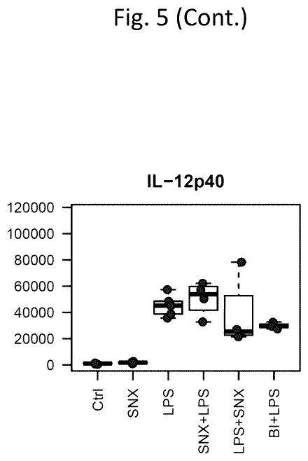

To verify that suppression of the induction of human pro-inflammatory cytokines is a general effect of CDK8/19 inhibition, we carried out another study in CIEA NOG-EXL mice, which differed from mice used in Example 2 in their average age (˜33 weeks, as opposed to 25-27 weeks in example 2) and having been engrafted with CD34+ hematopoietic stem cells 25-26 weeks rather than 21 weeks before the study. Mice were randomized into 6 groups (n=4-5), Group 1 being untreated control, Group 2 receiving 15u (a.k.a. SNX) (30 mg/kg p.o., administered as in example 2), Group 3 receiving LPS (1 mg/kg i.p.), Group 4 receiving LPS plus 15u administered 1 hr before LPS, Group 5 receiving LPS plus 15u administered 0.5 hr after LPS, and Group 6 receiving LPS plus CDK8/19 inhibitor BI-1347 (28) (10 mg/kg dissolved in 30% Propylene Glycol/70% PEG-400, p.o.) administered 1 hr before LPS. Mice were euthanized 6 hrs after LPS dosing and terminal blood samples were collected for cytokine analysis. As in Example 2, the effects of LPS and 15u on the expression of 48 human cytokines were measured using Human Cytokine 48-Plex Discovery Assay (Eve Technologies) using plasma samples at 1:100 dilution. The results of this analysis are shown in FIG. 5 for those cytokines that were induced by LPS. To assure more accurate assessment of effects of CDK8/19 inhibition on LPS-induced cytokines, 4 LPS-treated animals with low serum level (below 100 pg/mL) of IL-10, the cytokine whose LPS-mediated induction is not decreased by CDK8/19 inhibition, were excluded from the analysis (the low levels of cytokines in such animals are possibly due to insufficient administration of LPS). In this study, fewer cytokines (19 of 48) were induced by LPS than in Example 2, with many cytokines showing apparently higher expression levels in untreated mice possibly reflecting an inflammatory process that could have developed in humanized mice at the late time point (25-26 weeks) after the transplantation of CD34+ hematopoietic stem cells. Nevertheless, as in example 2, the induction of most of the LPS-induced cytokines was decreased by CDK8/19 inhibitor treatment (FIG. 5), including G-CSF, IFN-γ, IL-1 (α and β), IL-6, IL-8, MIG/CXCL9, MCP-1, MIP-1β and TNF (α and β) and IP-10. Among LPS-induced cytokines, only anti-inflammatory IL-10 was induced to a greater degree with than without CDK8/19 inhibitor treatment, an indication of anti-inflammatory activity. Remarkably, in most cases suppression of the induction of proinflammatory cytokines by 15u administered 0.5 hours after LPS was stronger than the effect of 15u administered 1 hour before LPS, indicating therapeutic benefit of administering CDK8/19 inhibitor to individuals that have been already exposed to an inflammation-inducing agent. Furthermore, both CDK8/19 inhibitors, SNX and BI-1347, suppressed the induction of the same proinflammatory cytokines (FIG. 5), indicating that such suppression is a general effect of chemically distinct CDK8/19 inhibitors.

### REFERENCES

1. J. B. Moore, C. H. June, Cytokine release syndrome in severe COVID-19. Science 368, 473-474 (2020).
2. R. M. Sterner, R. Sakemura, M. J. Cox, N. Yang, R. H. Khadka, C. L. Forsman, M. J. Hansen, F. Jin, K. Ayasoufi, M. Hefazi, K. J. Schick, D. K. Walters, O. Ahmed, D. Chappell, T. Sahmoud, C. Durrant, W. K. Nevala, M. M. Patnaik, L. R. Pease, K. E. Hedin, N. E. Kay, A. J. Johnson, S. S. Kenderian, GM-CSF inhibition reduces cytokine release syndrome and neuroinflammation but enhances CAR-T cell function in xenografts. Blood 133, 697-709 (2019).
3. T. N. Hoang, M. Pino, A. K. Boddapati, E. G. Viox, C. E. Starke, A. A. Upadhyay, S. Gumber, M. Nekorchuk, K. Busman-Sahay, Z. Strongin, J. L. Harper, G. K. Tharp, K. L. Pellegrini, S. Kirejczyk, K. Zandi, S. Tao, T. R. Horton, E. N. Beagle, E. A. Mahar, M. Y. H. Lee, J. Cohen, S. M. Jean, J. S. Wood, F. Connor-Stroud, R. L. Stammen, O. M. Delmas, S. Wang, K. A. Cooney, M. N. Sayegh, L. Wang, P. D. Filev, D. Weiskopf, G. Silvestri, J. Waggoner, A. Piantadosi, S. P. Kasturi, H. Al-Shakhshir, S. P. Ribeiro, R. P. Sekaly, R. D. Levit, J. D. Estes, T. H. Vanderford, R. F. Schinazi, S. E. Bosinger, M. Paiardini, Baricitinib treatment resolves lower-airway macrophage inflammation and neutrophil recruitment in SARS-COV-2-infected rhesus macaques. Cell 184, 460-475.e421 (2021).
4. J. Bennett, D. Capece, F. Begalli, D. Verzella, D. D' Andrea, L. Tornatore, G. Franzoso, NF-κB in the crosshairs: Rethinking an old riddle. The international journal of biochemistry & cell biology 95, 108-112 (2018).
5. C. B. Fant, D. J. Taatjes, Regulatory functions of the Mediator kinases CDK8 and CDK19. Transcription 10, 76-90 (2019).
6. J. Bancerek, Z. C. Poss, I. Steinparzer, V. Sedlyarov, T. Pfaffenwimmer, I. Mikulic, L. Dolken, B. Strobl, M. Muller, D. J. Taatjes, P. Kovarik, CDK8 kinase phosphorylates transcription factor STATI to selectively regulate the interferon response. Immunity 38, 250-262 (2013).
7. R. Firestein, A. J. Bass, S. Y. Kim, I. F. Dunn, S. J. Silver, I. Guney, E. Freed, A. H. Ligon, N. Vena, S. Ogino, M. G. Chheda, P. Tamayo, S. Finn, Y. Shrestha, J. S. Boehm, S. Jain, E. Bojarski, C. Mermel, J. Barretina, J. A. Chan, J. Baselga, J. Tabernero, D. E. Root, C. S. Fuchs, M. Loda, R. A. Shivdasani, M. Meyerson, W. C. Hahn, CDK8 is a colorectal cancer oncogene that regulates beta-catenin activity. Nature 455, 547-551 (2008).
8. C. Alarcon, A. I. Zaromytidou, Q. Xi, S. Gao, J. Yu, S. Fujisawa, A. Barlas, A. N. Miller, K. Manova-Todorova, M. J. Macias, G. Sapkota, D. Pan, J. Massague, Nuclear CDKs drive Smad transcriptional activation and turnover in BMP and TGF-beta pathways. Cell 139, 757-769 (2009).
9. A. Serrao, L. M. Jenkins, A. A. Chumanevich, B. Horst, J. Liang, M. L. Gatza, N. Y. Lee, I. B. Roninson, E. V. Broude, K. Mythreye, Mediator kinase CDK8/CDK19 drives YAP1-dependent BMP4-induced EMT in cancer. Oncogene 37, 4792-4808 (2018).
10. A. S. Adler, M. L. McCleland, T. Truong, S. Lau, Z. Modrusan, T. M. Soukup, M. Roose-Girma, E. M. Blackwood, R. Firestein, CDK8 maintains tumor dedifferentiation and embryonic stem cell pluripotency. Cancer Res 72, 2129-2139 (2012).
11. C. J. Fryer, J. B. White, K. A. Jones, Mastermind recruits CycC:CDK8 to phosphorylate the Notch ICD and coordinate activation with turnover. Mol. Cell 16, 509-520 (2004).
12. M. D. Galbraith, M. A. Allen, C. L. Bensard, X. Wang, M. K. Schwinn, B. Qin, H. W. Long, D. L. Daniels, W. C. Hahn, R. D. Dowell, J. M. Espinosa, HIFIA employs CDK8-mediator to stimulate RNAPII elongation in response to hypoxia. Cell 153, 1327-1339 (2013).
13. L. Johannessen, T. B. Sundberg, D. J. O'Connell, R. Kolde, J. Berstler, K. J. Billings, B. Khor, B. Seashore-Ludlow, A. Fassl, C. N. Russell, I. J. Latorre, B. Jiang, D. B. Graham, J. R. Perez, P. Sicinski, A. J. Phillips, S. L. Schreiber, N. S. Gray, A. F. Shamji, R. J. Xavier, Small-molecule studies identify CDK8 as a regulator of IL-10 in myeloid cells. Nat Chem Biol 13, 1102-+(2017).
14. M. S. McDermott, A. A. Chumanevich, C. U. Lim, J. Liang, M. Chen, S. Altilia, D. Oliver, J. M. Rae, M. Shtutman, H. Kiaris, B. Gyorffy, I. B. Roninson, E. V. Broude, Inhibition of CDK8 mediator kinase suppresses estrogen dependent transcription and the growth of estrogen receptor positive breast cancer. Oncotarget 8, 12558-12575 (2017).
15. M. Chen, J. Liang, H. Ji, Z. Yang, S. Altilia, B. Hu, A. Schronce, M. S. J. McDermott, G. P. Schools, C. U. Lim, D. Oliver, M. S. Shtutman, T. Lu, G. R. Stark, D. C. Porter, E. V. Broude, I. B. Roninson, CDK8/19 Mediator kinases potentiate induction of transcription by NFkappaB. Proc Natl Acad Sci USA 114, 10208-10213 (2017).
16. A. J. Donner, C. C. Ebmeier, D. J. Taatjes, J. M. Espinosa, CDK8 is a positive regulator of transcriptional elongation within the serum response network. Nat. Struct. Mol. Biol 17, 194-201 (2010).
17. I. Steinparzer, V. Sedlyarov, J. D. Rubin, K. Eislmayr, M. D. Galbraith, C. B. Levandowski, T. Vcelkova, L. Sneezum, F. Wascher, F. Amman, R. Kleinova, H. Bender, Z. Andrysik, J. M. Espinosa, G. Superti-Furga, R. D. Dowell, D. J. Taatjes, P. Kovarik, Transcriptional Responses to IFN-γ Require Mediator Kinase-Dependent Pause Release and Mechanistically Distinct CDK8 and CDK 19 Functions. Mol Cell 76, 485-499.e488 (2019).
18. T. Westerling, E. Kuuluvainen, T. P. Makela, Cdk8 is essential for preimplantation mouse development. Mol. Cell Biol 27, 6177-6182 (2007).
19. C. J. Lynch, R. Bernad, A. Martínez-Val, M. N. Shahbazi, S. Nóbrega-Pereira, I. Calvo, C. Blanco-Aparicio, C. Tarantino, E. Garreta, L. Richart-Ginés, N. Alcazar, O. Graña-Castro, G. Gómez-Lopez, I. Aksoy, M. Muñoz-Martín, S. Martinez, S. Ortega, S. Prieto, E. Simboeck, A. Camasses, C. Stephan-Otto Attolini, A. F. Fernandez, M. I. Sierra, M. F. Fraga, J. Pastor, D. Fisher, N. Montserrat, P. Savatier, J. Muñoz, M. Zernicka-Goetz, M. Serrano, Global hyperactivation of enhancers stabilizes human and mouse naive pluripotency through inhibition of CDK8/19 Mediator kinases. Nat Cell Biol 22, 1223-1238 (2020).
20. P. A. Clarke, M. J. Ortiz-Ruiz, R. TePoele, O. Adeniji-Popoola, G. Box, W. Court, S. Czasch, S. El Bawab, C. Esdar, K. Ewan, S. Gowan, A. De Haven Brandon, P. Hewitt, S. M. Hobbs, W. Kaufmann, A. Mallinger, F. Raynaud, T. Roe, F. Rohdich, K. Schiemann, S. Simon, R. Schneider, M. Valenti, S. Weigt, J. Blagg, A. Blaukat, T. C. Dale, S. A. Eccles, S. Hecht, K. Urbahns, P. Workman, D. Wienke, Assessing the mechanism and therapeutic potential of modulators of the human Mediator complex-associated protein kinases. Elife 5 (2016).
21. M. Chen, J. Li, J. Liang, Z. S. Thompson, K. Kathrein, E. V. Broude, I. B. Roninson, Systemic Toxicity Reported for CDK8/19 Inhibitors CCT251921 and MSC2530818 Is Not Due to Target Inhibition. Cells 8 (2019).
22. S. Yamamoto, T. Hagihara, Y. Horiuchi, A. Okui, S. Wani, T. Yoshida, T. Inoue, A. Tanaka, T. Ito, Y. Hirose, Y. Ohkuma, Mediator cyclin-dependent kinases upregulate transcription of inflammatory genes in cooperation with NF-kappaB and C/EBPbeta on stimulation of Toll-like receptor 9. Genes Cells 10.1111/gtc.12475 [doi] (2017).
23. C. B. Fant, D. J. Taatjes, Regulatory functions of the Mediator kinases CDK8 and CDK19. Transcription 10.1080/21541264.2018.1556915, 1-15 (2018).
24. S. Philip, M. Kumarasiri, T. Teo, M. Yu, S. Wang, Cyclin-Dependent Kinase 8: A New Hope in Targeted Cancer Therapy? J Med Chem 10.1021/acs.jmedchem.7b00901 (2018).
25. I. Menzl, A. Witalisz-Siepracka, V. Sexl, CDK8-Novel Therapeutic Opportunities. Pharmaceuticals (Basel) 12 (2019).
26. M. Xi, T. Chen, C. Wu, X. Gao, Y. Wu, X. Luo, K. Du, L. Yu, T. Cai, R. Shen, H. Sun, CDK8 as a therapeutic target for cancers and recent developments in discovery of CDK8 inhibitors. Eur J Med Chem 164, 77-91 (2019).
27. D. Ma, X. Chen, X. B. Shen, L. Q. Sheng, X. H. Liu, Binding patterns and structure-activity relationship of CDK8 inhibitors. Bioorg Chem 96, 103624 (2020).
28. M. H. Hofmann, R. Mani, H. Engelhardt, M. A. Impagnatiello, S. Carotta, M. Kerenyi, S. Lorenzo-Herrero, J. Böttcher, D. Scharn, H. Arnhof, A. Zoephel, R. Schnitzer, T. Gerstberger, M. P. Sanderson, G. Rajgolikar, S. Goswami, S. Vasu, P. Ettmayer, S. Gonzalez, M. Pearson, D. B. McConnell, N. Kraut, N. Muthusamy, J. Moll, Selective and Potent CDK8/19 Inhibitors Enhance NK-Cell Activity and Promote Tumor Surveillance. Mol Cancer Ther 19, 1018-1030 (2020).
29. M. Yu, T. Teo, Y. Yang, M. Li, Y. Long, S. Philip, B. Noll, G. K. Heinemann, S. Diab, P. Eldi, L. Mekonnen, A. T. Anshabo, M. H. Rahaman, R. Milne, J. D. Hayball, S. Wang, Potent and orally bioavailable CDK8 inhibitors: Design, synthesis, structure-activity relationship analysis and biological evaluation. Eur J Med Chem 214, 113248 (2021).
30. Q. Li, K. Feng, J. Liu, Y. Ren, Molecular modeling studies of novel naphthyridine and isoquinoline derivatives as CDK8 inhibitors. J Biomol Struct Dyn 10.1080/07391102.2020.1797537, 1-15 (2020).
31. S. Martínez-González, A. B. García, M. I. Albarrán, A. Cebriá, A. Amezquita-Alves, F. J. García-Campos, J. Martínez-Gago, J. Martínez-Torrecuadrada, I. Muñoz, C. Blanco-Aparicio, J. Pastor, Pyrido[2,3-b][1,5]benzoxazepin-5(6H)-one derivatives as CDK8 inhibitors. Eur J Med Chem 201, 112443 (2020).
32. E. Solum, T. V. Hansen, R. Aesoy, L. Herfindal, New CDK8 inhibitors as potential anti-leukemic agents—Design, synthesis and biological evaluation. Bioorganic & medicinal chemistry 28, 115461 (2020).
33. M. M. Al-Sanea, Synthesis and biological evaluation of small molecule modulators of CDK8/Cyclin C complex with phenylaminoquinoline scaffold. PeerJ 8, e8649 (2020).
34. J. M. Grandjean, A. Y. Jiu, J. W. West, A. Aoyagi, D. G. Droege, M. Elepano, M. Hirasawa, M. Hirouchi, R. Murakami, J. Lee, K. Sasaki, S. Hirano, T. Ohyama, B. C. Tang, R. J. Vaz, M. Inoue, S. H. Olson, S. B. Prusiner, J. Conrad, N. A. Paras, Discovery of 4-Piperazine Isoquinoline Derivatives as Potent and Brain-Permeable Tau Prion Inhibitors with CDK8 Activity. ACS Med Chem Lett 11, 127-132 (2020).
35. M. J. Ramos-Benitez, C. Ruiz-Jimenez, J. J. Rosado-Franco, W. D. Ramos-Pérez, L. B. Mendez, A. Osuna, A. M. Espino, Fh15 Blocks the Lipopolysaccharide-Induced Cytokine Storm While Modulating Peritoneal Macrophage Migration and CD38 Expression within Spleen Macrophages in a Mouse Model of Septic Shock. mSphere 3 (2018).
36. C. Chen, X. Li, C. Li, J. Jin, D. Wang, Y. Zhao, Y. Gu, M. Chen, S. Zhu, H. Liu, T. Lv, F. Zhang, Y. Song, CD39(+) Regulatory T Cells Attenuate Lipopolysaccharide-Induced Acute Lung Injury via Autophagy and the ERK/FOS Pathway. Front Immunol 11, 602605 (2020).
37. L. Mazgaeen, P. Gurung, Recent Advances in Lipopolysaccharide Recognition Systems. Int J Mol Sci 21 (2020).
38. J. Zhou, H. Li, X. Xia, A. Herrera, N. Pollock, V. Reebye, M. H. Sodergren, S. Dorman, B. H. Littman, D. Doogan, K. W. Huang, R. Habib, D. Blakey, N. A. Habib, J. J. Rossi, Anti-inflammatory Activity of MTL-CEBPA, a Small Activating RNA Drug, in LPS-Stimulated Monocytes and Humanized Mice. Mol Ther 27, 999-1016 (2019).

1. A method for treating a subject in need of a treatment for a cytokine storm or elevated amounts of a multiplicity of different cytokine-storm mediating cytokines, the method comprising administering an effective amount of an inhibitor of CDK8 and CDK 19 or a pharmaceutical composition comprising the effective amount of the inhibitor of CDK8 and CDK19 to the subject.

2. The method of claim 1, wherein the inhibitor of CDK8 and CDK19 is 3-amino-4-(4-(4 (dimethylcarbamoyl) phenyl)-1,4-diazepan-1-yl)thieno[2,3-b]pyridine-2-carboxamide (15u) or 2-(4-(4-(isoquinolin-4-yl)phenyl)-1H-pyrazol-1-yl)-N,N-dimethylacetamide (BI-1347).

3. (canceled)

4. The method of claim 1, wherein the inhibitor of CDK8 and CDK19 selectively inhibits CDK8 and CDK19.

5. The method of claim 1, wherein elevated amounts of a multiplicity of different cytokines in the subject are induced by a pathogen.

6. (canceled)

7. (canceled)

8. (canceled)

9. The method of claim 1, wherein elevated amounts of a multiplicity of different cytokines in the subject are induced by a cancer.

10. The method of claim 1, wherein elevated amounts of a multiplicity of different cytokines in the subject are induced by an autoimmune condition.

11. The method of claim 1, wherein elevated amounts of a multiplicity of different cytokines in the subject are induced by an immunotherapy.

12. The method of claim 1, wherein the subject is in need of a treatment for acute respiratory distress syndrome, hypoxemia, acute systemic inflammation, or secondary organ dysfunction.

13. (canceled)

14. (canceled)

15. (canceled)

16. The method of claim 1, wherein the subject is a human.

17. The method of claim 1, wherein the effective amount of the inhibitor of CDK8 and CDK 19 reduces two or more different cytokine-storm mediating cytokines.

18. The method of claim 17, wherein the two or more different cytokine-storm mediating cytokines are selected from IL-6, TNFα, GM-CSF, IFN-γ, IL-1α, IL-1β, IL-8, IL-12 (p40), IL-12 (p70), IL-18, MIG/CXCL9, MIP-1α, MIP-1β, and TNFβ.

19. (canceled)

20. (canceled)

21. (canceled)

22. The method of claim 1, wherein the effective amount of the inhibitor of CDK8 and CDK19 does not significantly reduce the amount of an anti-inflammatory cytokine.

23. The method of claim 22, wherein the anti-inflammatory cytokine is IL-10.

24. (canceled)

25. (canceled)

26. The method of claim 1, wherein the effective amount of the inhibitor of CDK8 and CDK 19 reduces RNA expression of two or more different cytokine-storm mediating cytokines.

27. (canceled)

28. (canceled)

29. (canceled)

30. (canceled)

31. The method of claim 1, wherein the effective amount of the inhibitor of CDK8 and CDK19 is administered prior to induction of elevated amounts of a multiplicity of different cytokines in the subject.

32. The method of claim 1, wherein the effective amount of the inhibitor of CDK8 and CDK19 is administered after induction of elevated amounts of the multiplicity of different cytokines in the subject.

33. The method of claim 1, wherein the subject is in need of treatment for the cytokine storm.

34. The method of claim 1, wherein the subject is in need of the treatment for elevated amounts of the multiplicity of different cytokine-storm mediating cytokines.

35. A method for treating a subject in need of a treatment for a cytokine storm or elevated amounts of a multiplicity of different cytokine-storm mediating cytokines, the method comprising detecting two or more different cytokine-storm mediating cytokines or RNA expression thereof in a sample obtained from the subject and administering the effective amount of the inhibitor of CDK8 and CDK 19 according to claim 1 to the subject if the subject has elevated amounts of the two or more different cytokine-storm mediating cytokines or RNA expression thereof.

36. A method for identifying patients in need a treatment for a cytokine storm or elevated amounts of a multiplicity of different cytokine-storm mediating cytokines, the method comprising detecting for elevated amounts of two or more different cytokine-storm mediating cytokines or RNA expression thereof in a sample from the subject, wherein the subject is identified for treatment with the effective amount of the inhibitor of CDK8 and CDK19 according to claim 1 if the subject has elevated amounts of the two or more different cytokine-storm mediating cytokines or RNA expression thereof.

37. (canceled)

38. (canceled)

39. (canceled)

40. (canceled)
